(19) United States
(12) Patent Application Publication
(10) Pub. No.: US 2005/0130214 A1
(43) Pub. Date: Jun. 16, 2005

(54) Molecular binding event detection using separation channels

(75) Inventors: David K. Biegelsen, Portola Valley, CA (US); Robert A. Street, Palo Alto, CA (US)

Correspondence Address: BEVER, HOFFMAN & HARMS, LLP, 1432 CONCANNON BLVD, BLDG G, LIVERMORE, CA, 64550-6006, US

(73) Assignee: Xerox Corporation

(21) Appl. No.: 11/015,827

(22) Filed: Dec. 17, 2004

Related U.S. Application Data
(62) Division of application No. 10/737,692, filed on Dec. 15, 2003.

(51) Int. Cl. C12Q 1/68; G01N 33/53; G06F 19/00; G01N 33/48; G01N 33/50; G01N 33/558

(52) U.S. Cl. 435/6; 435/7.1; 702/19

(57) ABSTRACT

Detecting binding events between first and second molecules (e.g., ligands and proteins) includes mixing at the first end of a test channel, then separating the bound/unbound molecules (e.g., using electrophoresis) by causing the molecules to move down the channel such that groups of bound/unbound molecules move along the channel at different rates. The groups are then detected, measured and compared against established reference data to determine whether a binding event has occurred. A reference channel is utilized to provide reference data and to identify unbound molecule groups. Radiant energy and a bolometer are utilized to measure the molecule groups

### RELATED APPLICATION

This application is a divisional of U.S. patent application Ser. No. 10/737,692, entitled “Molecular Binding Event Detection Using Separation Channels”, filed Dec. 15, 2003.

### FIELD OF THE INVENTION

The present invention is related to biomedical testing systems and methods, and in particular to systems and methods for detecting binding events between two molecules.

### BACKGROUND OF THE INVENTION

The detection of binding events between two organic molecules is an important issue in biological studies and drug discovery. There seem to be no generic (i.e., independent of the specific molecules involved in the binding process) and inexpensive methods for detecting molecular binding, much less methods for fabricating arrays that can be used to assay many thousands of possible binding pairs in parallel.

Proteomics represents one branch of biological studies in which the detection of binding events is particularly important at this time. Proteomics involves the use of various techniques to analyze the structure, function, and interactions of proteins in order to, for example, identify and generate new drugs. Recent achievements in genetic research have identified a large number of previously unknown proteins whose function and structure are believed to be extremely important in drug discovery. Deciphering the structures and functions of unknown entities (e.g., proteins) is possible by detecting their interaction (i.e., ability to bind) with known ligand entities. Accordingly, given the extremely large number of unknown proteins and possible protein/ligand combinations that could yield valuable drugs, the need for an inexpensive method and apparatus for detecting binding events between proteins and ligands is particularly important.

What is needed is a generic and inexpensive method for detecting molecular binding events, and an apparatus that facilitates this method in a reliable manner using very small (e.g., sub-nanoliter) molecule doses. What is also needed is such an apparatus and method that is able to assay thousands of possible binding pairs in parallel. What is also needed is an apparatus and method that is able to provide quantitative binding kinetics information.

### SUMMARY OF THE INVENTION

The present invention is directed to a method and apparatus for detecting binding events between two or more molecules (e.g., a ligand and a protein) that includes mixing the molecules at a first location in a test channel, separating the bound/unbound molecules (e.g., using electrophoresis) such that groups of bound and unbound molecules move along the channel at different rates, detecting and measuring the size of the bound/unbound molecule groups, and then comparing the measurement values against established reference data to determine whether a binding event has occurred. Mixing involves, for example, injecting sub-nanoliter-sized doses of a selected ligand and a selected protein into a receptor well located at a first end of the test channel, and activating a suitable mixing mechanism. Separating involves, for example, applying a suitable motive force (e.g., an electric field) that causes the bound and unbound molecules to separate into three possible groups that move along the channel at different rates: the smaller unbound ligands may, for example, form a first (fastest) group in the channel, followed by the larger unbound proteins, and then the bound ligand/protein pairs. The actual magnitudes and sign of dispersed molecular velocities depends on the particular channel structure, channel filling (e.g. particle packing, gel, empty, etc.), motive mechanism, molecular properties

(e.g. charge, mass, size, state of naturation, etc.)

Detection and measurement of the size of each group (i.e., an estimate of the number of molecules in each group) is performed using a stationary detector (e.g., a bolometer) that is positioned at a second location along the test channel. Finally, these measurements are then compared with reference data to determine whether a binding event has occurred, and can be used to estimate the relative strength of the binding event. For example, in one embodiment, the detection of two relatively large groups passing the detector may be interpreted as groups of unbound ligands and unbound proteins, thereby indicating a non-binding event. In contrast, two smaller groups followed by a larger group, or a single large group may be interpreted to indicate moderate to strong binding between the proteins and ligands. Accordingly, the present invention provides a generic and inexpensive method for detecting molecular binding events.

According to an embodiment of the present invention, photothermal detection is utilized to measure extremely small (e.g., sub-nanoliter) doses of the bound/unbound molecular groups moving in the test channel. In one embodiment, a radiant energy source is transmitted into the test channel at a wavelength that is absorbed by the moving molecules, but is not significantly absorbed by the channel liquid (e.g., water) in which the molecules are suspended. To further enhance optical absorption by the molecules, the radiant energy is repeatedly passed through the channel using a reflecting device (e.g., an etalon). The optically absorbed energy is converted to heat by the molecules and dissipated in the liquid. A highly sensitive thermometer (e.g., a bolometer) is positioned in the channel and utilized to generate temperature profiles indicating local heating of the channel liquid as the groups of bound and unbound molecules pass through. The temperature profiles are then analyzed (e.g., compared with reference data) to determine whether a binding event has taken place. Accordingly, the present invention facilitates binding event detection using very small (e.g., sub-nanoliter) molecule doses.

According to another embodiment, an apparatus for detecting binding events utilizes both a test (first) channel and a reference (second) channel or channels that are substantially identical in size and length, subjected to the same molecular moving force (e.g., an electric field), and are coupled to similar detectors. The test channel receives the mixture of first and second molecules (e.g., a ligand and a protein), whereas the reference channel only receives a dose of the first molecule (e.g., the ligand). After a suitable mixing period, the electric field is applied to both channels that causes the ligands to travel down the reference channel, and causes free ligands (if present) to separate and travel down the test channel. A reference ligand measurement is generated when ligands subsequently pass the reference channel detector. This reference measurement both indicates when free ligands (if present) will pass the detector in the test channel (i.e., either simultaneously or after a predicable delay associated with the separation process), and indicates an approximate free ligand measurement that would indicate a non-binding event has occurred. That is, a minimal difference between the reference channel and test channel measurements indicates a large number of free (unbound) ligands in the test channel, thereby indicating that a non-binding event has occurred. Conversely, a significant difference between the reference channel and test channel measurements indicates a small number of free ligands in the test channel, thereby indicating that a binding event has occurred. Accordingly, by coordinating a reference channel measurement with the test channel measurement, the present invention provides a very sensitive, reliable and inexpensive method for detecting binding events and can be used even if reference data for a particular ligand is unavailable. Moreover, running the two or more channels under nominally identical conditions provides high common mode rejection of noise and mitigates the need to tightly control the test parameters such as temperature, absolute concentration, electric field, pH, etc.

According to another embodiment, a batch-fabricated fluidic system for handling large numbers of ligands and/or proteins in parallel is utilized with multiple channels for detecting binding/non-binding on a massively parallel scale. In one specific embodiment, multiple pairs of channels are provided in parallel, with each pair of channels receiving a specified ligand and a subject protein, and each channel pair operating as described above to detect binding events. With this arrangement, binding events are performed on a massively parallel basis. In another specific embodiment, multiple inlet ports selectively inject proteins and ligands into a single pair of channels that is flushed after each test, thereby facilitating systematic binding event detection while minimizing the need for broad-based detection and analysis systems.

In accordance with another aspect of the present invention, relatively high throughput is achieved by mixing two or more non-interacting second molecules (e.g., ligands) with the first molecule (e.g., the subject protein) in the single-channel and two-channel apparatus discussed above. If one of the two or more ligands binds with the protein, then the resulting absence of the binding ligand is detected using the methods described above, and one or more additional separation processes can be used to identify the specific binding ligand (if necessary). By testing multiple ligands in each channel, the number of test iterations required to identify a relatively small number of binding ligands from a relatively large library of ligands can be significantly reduced. We note that different ligands can have different spectral dependence for their optical absorptions and so can be distinguished if the illumination wavelengths are selected from the total available spectrum.

### BRIEF DESCRIPTION OF THE DRAWINGS

These and other features, aspects and advantages of the present invention will become better understood with regard to the following description, appended claims, and accompanying drawings, where:

FIG. 1 is a simplified schematic diagram depicting an apparatus for detecting binding events according to an embodiment of the present invention;

FIG. 2 is a flow diagram showing a generalized method for detecting binding events between two or more molecules according to another embodiment of the present invention;

FIGS. 3(A), 3(B), 3(C), 3(D), 3(E) and 3(F) are simplified diagrams depicting portions of a binding event detection apparatus according to another embodiment of the present invention;

FIG. 4 is a simplified cross-sectional side view showing a portion of the apparatus shown in FIG. 3(E);

FIGS. 5(A), 5(B) and 5(C) are diagrams illustrating a portion of the apparatus shown in FIG. 3(E) and depict a group of molecules passing a heat measuring probe according to another embodiment of the present invention;

FIGS. 6(A), 6(B) and 6(C) are graphs illustrating the generation of a temperature profile generated by the heat measuring probe in response to the molecule group illustrated in FIGS. 5(A) through 5 (C);

FIGS. 7(A), 7(B) and 7(C) are diagrams illustrating a portion of the apparatus shown in FIG. 3(E) depicting groups of molecules passing a heat measuring probe according to another embodiment of the present invention;

FIGS. 8(A), 8(B) and 8(C) are graphs illustrating the generation of a series of temperature profiles generated by the heat measuring probe in response to the molecule groups illustrated in FIGS. 7(A) through 7(C);

FIG. 9 is a simplified schematic diagram depicting an apparatus for detecting binding events according to another embodiment of the present invention;

FIG. 10 is a flow diagram showing a generalized method for detecting binding events according to another embodiment of the present invention;

FIGS. 11(A), 11(B), 11(C) and 11(D) are simplified diagrams depicting portions of a binding event detection apparatus during the binding event detection method of FIG. 10 according to another embodiment of the present invention;

FIGS. 12(A) and 12(B) are graphs illustrating temperature profiles associated with the molecule groups illustrated in FIG. 11(C);

FIGS. 13(A) and 13(B) are graphs illustrating temperature profiles associated with the molecule groups illustrated in FIG. 11(D);

FIG. 14 is a simplified diagram showing an apparatus for detecting binding events according to another specific embodiment;

FIG. 15 is a simplified diagram showing an apparatus for detecting binding events according to another specific embodiment; and

FIGS. 16(A) and 16(B) are simplified diagrams depicting a method for detecting binding events according to another aspect of the present invention.

### DETAILED DESCRIPTION OF THE DRAWINGS

The present invention is described below with specific reference to binding events involving a selected ligand/protein pair. The use of ligand/protein pairs is intended to be exemplary, and the methods and apparatus described herein may be used to detect binding events between other molecule types, and further may be expanded to detect binding events involving three or more molecule types. Moreover, the components and processes described herein with reference to certain specific embodiments are intended to be exemplary, and not intended to be limiting unless otherwise specified in the appended claims.

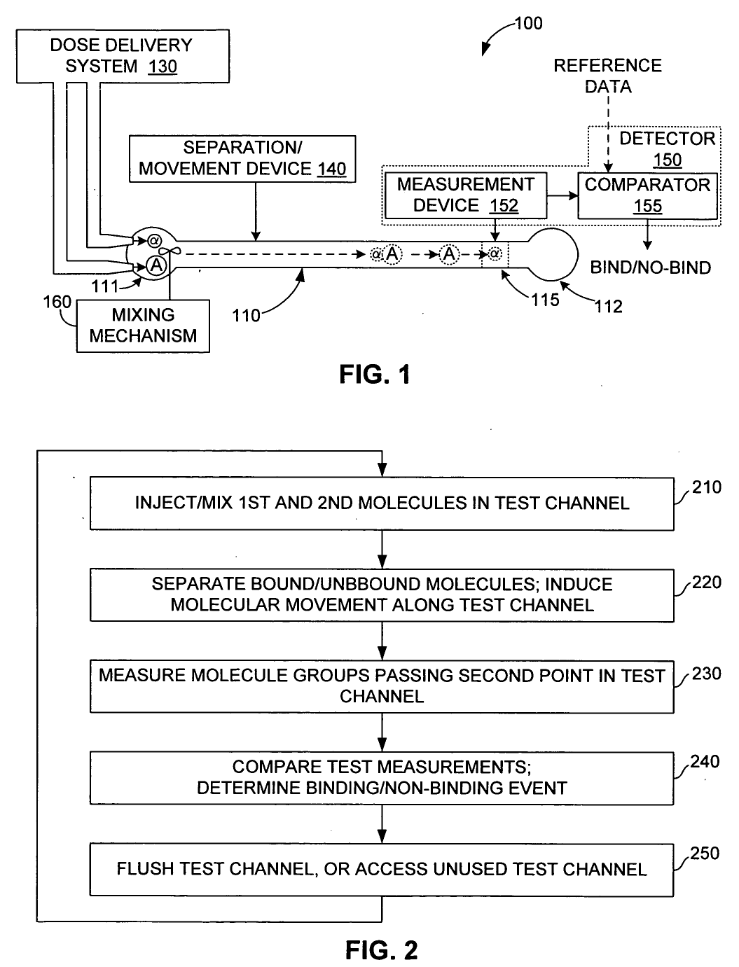

FIG. 1 is a simplified schematic diagram depicting an apparatus 100 for detecting binding events between a ligand (first molecule) a and a protein (second molecule) A according to a simplified embodiment of the present invention. Apparatus 100 generally includes a test channel 110, an optional dose delivery system 130, a molecular separation movement-inducing device 140, and a detection device (detector) 150.

In one embodiment, test channel 110 represents one of several similar microchannels forming a microfluidics environment that is fabricated, for example, on a substrate in accordance with conventional methods. In one specific embodiment, test channel 110 is fabricated using one of various known batch fabrication techniques, such as etching glass, embossing in plastic, or photolithographic patterning in SU-8. In a specific embodiment, test channel 110 is a microchannel structure that is approximately 0.1-to 50 mm in length and has a depth and width of approximately 0.1 to 1.0 mm. Located at a first end (location) of test channel 110 is a receptor well 111 that serves as a mixing point for the ligands and proteins. Located at the opposite end of test channel 110 is a collection area or sump 112 that receives molecules that have moved through channel 110, and communicates with an optional exit point through which these molecules can be removed from channel 110. Test channel 110 contains a suitable channel fluid (e.g., de-ionized water) that facilitates molecular separation/movement in the manner described below.

In one embodiment of the present invention, the ligand/protein mixing process involves injecting or otherwise transporting predefined doses (e.g., a selected ligand a or a subject protein A) into receptor well 111 using a dose delivery system 130, and then stimulating the binding process using a suitable mixing mechanism 160. Delivery system 130 consists of a suitable liquid transport and distribution system, similar to an ink supply mechanism utilized in an inkjet printer, an array of micropipettes, etc., that is capable of transporting sub-nanoliter doses to receptor well 111 using well-established techniques. Those skilled in the art will recognize other fluidic plumbing arrangements may also be utilized to transport the ligand and/or protein doses to channel 110, and the distribution may be performed manually (as opposed to automatically). Mixing mechanism 160 functions to interdiffuse, wrap flow fields, heat, agitate or otherwise intermix ligands a and proteins A in a manner that promotes binding.

Molecular separation/movement device 140 functions to apply a suitable motive force that induces movement of unbound proteins A and ligands α, or bound protein/ligand pairs, along test channel 110 at different rates based, for example, on molecular size. When applied to a mixture including relatively small ligands a and relatively large proteins A, this motive force causes separation into three groups (assuming some but not all ligand/protein pairs bind together) that move along test channel 110 from receptor well 111 to sump 112. For example, in a capillary electrophoresis (EP) configuration an electric field is applied across the length of the channel and molecules are moved one way or the other at a velocity proportional to their charge and inversely proportional to their mass and size. In the case of electro-osmotic flow (EOF) in an open channel all molecules, independent of charge and mass are carried by the ionic water at the same rate (no dispersion.) However, if the channel is filled totally or throughout a short segment of the channel with micro- or nano-beads, membrane or gel, creating a porous frit or sieve, the EOF sweeps the molecules along. However, smaller molecules tend to diffuse into nano-pockets wherein they dwell longer than larger molecules which are less likely to find their way into such small regions. Therefore, the larger molecules arrive downstream earlier, the opposite of the dispersion in the EP case. See, e.g., DNA size separation using artificially nanostructured matrix, M. Baba, T. Sano, N. Iguchi, K. Iida, T. Sakamoto, and H. Kawaura, Applied Physics Letters Vol 83(7) pp. 1468-1470, Aug. 18, 2003. Non-zero molecular charge causes both an EOF and EP mechanism to act simultaneously on the molecular velocity, either in additive or subtractive manners. In any event, dispersion separates the molecules according to their specific properties so that downstream detection can differentiate and identify separated molecular components. Thus, in the EP case, the smaller unbound ligands a tend to form a first (fastest) group moving along channel 110, followed by the larger unbound proteins A, and then the bound ligand/protein pairs. As described in additional detail below, such movement is induced, for example, by electrophoresis (i.e., applying an electric field such that molecules move through the stationary channel fluid provided in channel 110). In other embodiments, suitable movement is generated, for example, by electrokinetically pumping the liquid in channel 110, thereby sweeping along the molecules in the fluid flow, with interactions between the molecules and the walls of the channel causing the larger, heavier molecules to be delayed relative to smaller, lighter molecules. Similarly, pressure or centrifuge-induced flows through gel packed channels can disperse the molecules by mass and independent of charge. Other molecular separation/movement device 140 can also be utilized.

As indicated on the right side of FIG. 1, detection device 150 includes a measurement device 152 and a comparator 155.

Measurement device 152 is positioned at a predetermined (second) location 115 to detect the groups of molecules as they move along channel 110 from receptor well 111 toward sump 112. Note that the length and diameter of channel 110 and the position of location 115 are selected to allow adequate separation of the bound/unbound groups. Measurement device 152 utilizes, for example, photothermal or optical methods to detect the size of (and, in effect, the number of molecules in) each group as it passes location 115. Device 152 can be a semiconductor, resistor, or microelectromechanical bolometer, an optical deflection sensor using a split photodiode, or simply a photodiode to measure total optical transmission. Transmission measurements attempt to sense a small decrease in a large background. The photothermal effect is preferred here because it measures a small increase on a small background, and only when molecules of interest are present. Using filters or active dispersion or multiple light sources the spectral dependence of the photothermal response of different molecules can be used to disambiguate signals in the rare cases that multiple ligand bands overlap.

Comparator 155 (e.g., an application specific logic circuit, or a general purpose microcomputer or workstation) receives measurement data from measurement device 152, and compares the measured values from test channel 110 with reference data representing a known binding event or non-binding event. As indicated in FIG. 1, the reference data may be either supplied from reference data, both arrival time and magnitude, from an external source (e.g., previously established data that is entered into comparator), or previously established measurement values that are stored in the comparator. Based on this comparison, using the methods described below, the present invention facilitates determining whether a binding event has occurred in a reliable and economical manner.

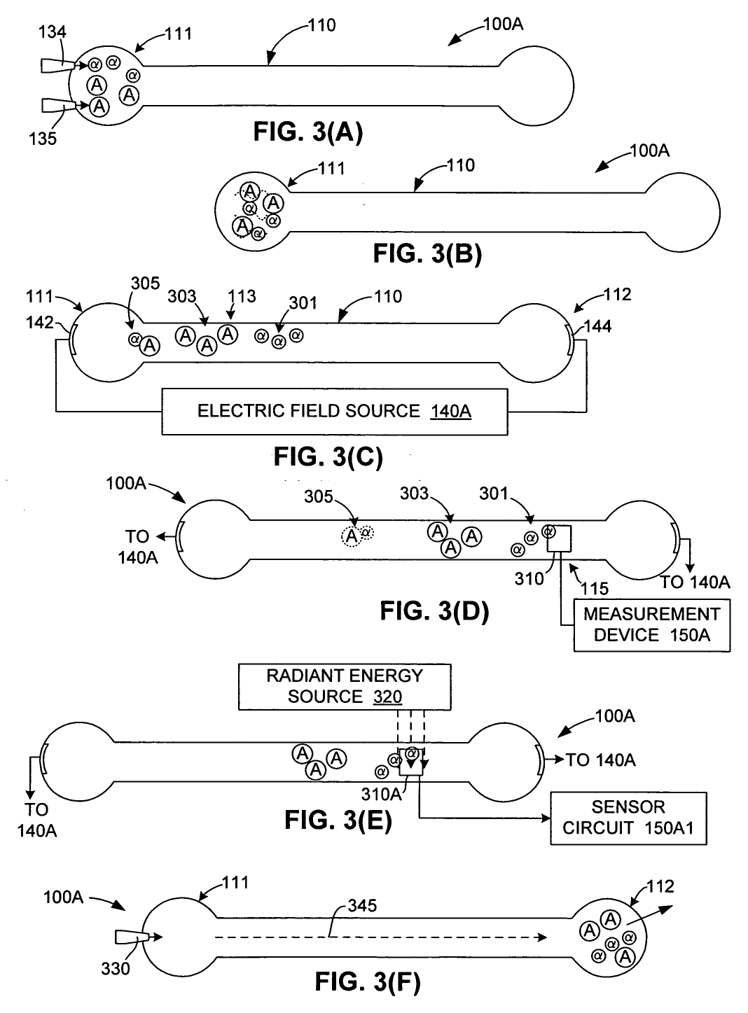

FIG. 2 is a flow diagram showing a generalized method for detecting binding events between two or more molecules according to an embodiment of the present invention. For convenience, the method depicted in the flow diagram of FIG. 2 is described below with reference to FIGS. 3(A) through 3(F) that depict portions of an apparatus 100A, which represents a specific embodiment of apparatus 100 (FIG. 1). Note that the method shown in FIG. 2 is not necessarily restricted to apparatus 100A.

Referring to the upper portion of FIG. 2, FIG. 3(A) and FIG. 3(B), the method begins by injecting and/or mixing first and second molecules (e.g., ligands and proteins) in a first location of test channel 110 (block 210). In one embodiment, this injection process involves transporting and injecting ligands a and proteins A into receptor well 111 using mechanisms such as those described above with reference to FIG. 1. In alternative embodiments, one or both of ligands a and proteins A may be pre-positioned or otherwise placed in receptor well 111. The dose sizes placed in receptor well 111 determined according to well-established techniques. Generally it is desirable to have the ligand concentration be much less than the protein concentration so that reaction goes to completion. In fact an important use of the present invention would be to vary the ratio of α to A concentrations from small to approximately equal to measure rate constants. Referring to FIG. 3(B), the first and second molecules are then mixed using a suitable mixing process (e.g., swirling flow induced by EOF across a patterned wall 160A). In addition to this mixing process, other known procedures may be utilized to promote mixing of the first and second molecules, such as the thermally induced Couette flows or activated interdiffusion, direct interdiffusion in the co-dispensed liquid droplets, acoustic streaming initiated by an acoustic source, etc.

Referring again to FIG. 2 and FIG. 3(C), the mixed molecules are then induced to move down the test channel and become separated by type (block 220). As indicated in FIG. 3(C), in one embodiment, electrophoretic separation is initiated by activating a suitable electric field source 140A coupled to electrodes 142 and 144, which are located at opposite ends of test channel 110. The resulting electric field causes unbound ligands α to separate from unbound proteins A and bound protein/ligand pairs (if present) such that the unbound ligands a move substantially as a group 301 through an intermediate section 113 of channel 110 toward sump 112 at a relatively fast (first) rate, followed by a second group 303 including unbound proteins A moving at a somewhat slower (second) rate, and finally a third group 305 including bound protein/ligand pairs (if present) at a third (e.g., slower) rate. As discussed above, separation by dispersive movement of ligands a and proteins A in this manner can also be produced, for example, by generating a suitable flow through test channel 110. As described above, a preferred method that is nearly universally applicable—because it does not depend on sign or magnitude of molecular charge to provide the motive force—is EOF pumping through a sieving channel.

As indicated in block 230 of FIG. 2, and referring to FIG. 3(D), the moving molecules are then measured using, for example, a stationary probe 310 that is located at second location 115 of channel 110 and is coupled to a suitable measurement device (e.g., a temperature dependent resistor or diode) 150A.

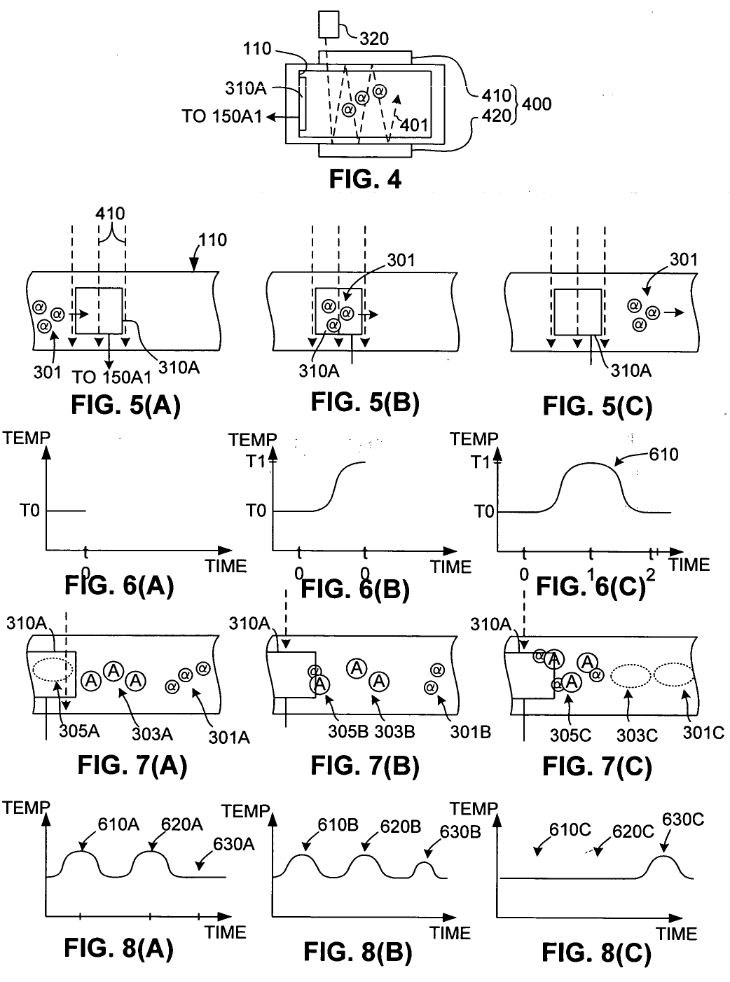

FIGS. 3(E) and 4 show a specific embodiment of the arrangement shown in FIG. 3(D) in which a radiant energy source 320 is positioned to transmit modulated illumination into test channel 110, and in which a thermal sensor probe 310A includes a thin film theromocouple or other bolometric sensor that is coupled to a sensor circuit 150A1. Radiant energy source 320 transmits modulated illumination having a wavelength of approximately 210 nm to 250 nm (which is a part of the spectrum where water is relatively transparent but most ligands and proteins absorb), and thermal sensor probe 310A is maintained in close thermal contact with the fluid (e.g., water) contained in test channel 110 to detect absorption of the modulated illumination (i.e., temperature changes in the channel fluid adjacent probe 310A) by ligands a and proteins A using lock-in detection (i.e., photothermal detection). As indicated in FIG. 4, thermal sensor probe 310A is arranged to be located outside of the illumination beam, i.e., along or on the walls forming channel 110, and parallel to the direction of incidence of the optical beam. Alternatively the sensor probe can be protected from optical absorption by a reflective coating. In the embodiment indicated in FIG. 4, an etalon 400 is utilized to reflect the radiation within channel 110. Etalon 400 includes a partially transparent mirror portion 410 and a totally reflecting mirror portion 420 respectively located above and below channel 110 to repeatedly reflect the radiated energy beams 401 (one shown for clarity) emitted by source 320. Mirror portions 410 and 420 can be deposited metal films or dielectric multilayer stacks. Because all non-conjugated organic molecules strongly absorb radiated energy having a wavelength of approximately 210 nm to 250 nm, and because water is relatively non-absorbing between 200 nm and 450 nm, this photothermal form of detection is quite sensitive, general, and independent of the specific molecular structure of ligands α and proteins A. In contrast to the photothermal approach, a usual absorption method utilizes a sensor that is located in the radiated beam that senses the passing molecules by small reductions in the radiated energy (i.e., by detecting “shadows” cast by the molecules). Not only is the photothermal detection method more sensitive than this absorption method (i.e., because photothermal detection measures only the energy that is absorbed), it provides an added bonus in that, because of the phase sensitive detection, photothermal detection is insensitive to incoherent background heat. Sensor circuit 150A1 produces a thermal profile for each group of molecules passing probe 310 in the manner described below with reference to FIGS. 5(A) through 8(C).

FIGS. 5(A) through 5(C) illustrate a portion of channel 110 adjacent location 115 as a group 301 of ligands a pass probe 310A during a first period of time t0 to t2. FIGS. 6(A) through 6(C) depict an idealized thermal profile generated, for example, by probe 310A and sensor circuit 150A1 (FIG. 3(E)) during this time period. FIG. 5(A) illustrates group 301 as it approaches probe 310A at time t0, and FIG. 6(A) indicates that the temperature of the fluid adjacent to probe 310A at this point in the measurement process remains relatively constant at temperature T0. Note that modulated illumination 401 passing through test channel 100 are partially absorbed by ligands α, which in turn radiate heat into the surrounding fluid (as indicated by short lines extending from ligands a). However, as discussed above, due to the absence of heat absorbing material adjacent probe 310A, the measured temperature remains relatively low. FIG. 5(B) illustrates group 301 at time t1 as it passes probe 310A, thereby heating the channel fluid located adjacent to probe 310A. As shown in FIG. 6(B), this heating causes the measured temperature to increase to temperature T1. Finally, FIG. 5(C) illustrates group 301 at a time t2 as it moves away from probe 310A. As indicated by completed temperature profile 610 depicted in FIG. 6(C), the resulting absence of heat absorbing material causes the temperature of the fluid adjacent to probe 310A to gradually return to temperature T0. Note that FIGS. 6(A) through 6(C) depict an idealized temperature profile, and that the measured temperature may not immediately return to starting temperature T0 after group 301 passes. The temperature rise is proportional to the number density and type of molecules present.

Referring again to FIG. 2, after measuring one or more groups of molecules, these measurements are utilized to determine whether a binding event has occurred, or whether ligands a have failed to bind with proteins A (i.e., a non-binding event).

According to one embodiment, the temperature profile of one or more molecule groups is/are compared with externally-supplied or otherwise predetermined reference data to determine whether a binding event has occurred between ligands a and proteins A. For example, utilizing temperature profile 610 (FIG. 6(C)), maximum temperature T1 may be compared with an experimentally produced temperature, which is produced under non-binding conditions, to determine that substantially all ligands a remained unbound, thereby indicating a weak or non-binding event. Conversely, if temperature T1 is substantially lower than an experimentally generated temperature indicative of a non-binding event, then a “binding event” detection message is generated. Note that maximum temperature is used in this example for brevity, and those skilled in the art will recognize that such comparisons are more reliably performed using intermediate measured and calculated measurement values.

A second approach for determining the occurrence of binding events is now described with reference to FIGS. 7(A) to 8(C), where FIGS. 7(A) through 7(C) are simplified diagrams depicting various combinations of bound and unbound molecules, and FIGS. 8(A) through 8(C) are graphs indicating various temperature profiles generated by the combinations of FIGS. 7(A) through 7(C), respectively. According to this approach, as set forth in the following examples, at least two of the three thermal profiles generated during the measurement process are compared to determine whether a binding event has occurred.

A first example is indicated in FIG. 7(A), where a non-binding event produces a relatively large unbound ligand group 301A, a relatively large unbound protein group 303A, and an empty bound ligand/protein group 305A (indicated by the empty dashed oval). The resulting thermal profiles are indicated in FIG. 8(A), where the relatively large unbound ligand group generates a relatively strong thermal profile 610A, and the relatively large unbound protein group generates a relatively strong thermal profile 620A in the manner described above. Note that the empty bound ligand/protein group generates a flat thermal profile 630A. The generation of two detectable thermal profile is indicative that substantially none of the ligands and proteins are involved in bound pairs, thereby indicating a non-binding event.

A second example is indicated in FIG. 7(B), where a weak binding event produces a moderate-sized unbound ligand group 301B, a moderate-sized unbound protein group 303B, and a small bound ligand/protein group 305B (indicated by one bound pair. The resulting thermal profiles are indicated in FIG. 8(B), where the moderate-sized unbound ligand group generates a moderate thermal profile 610B, the moderate unbound protein group generates a moderate thermal profile 620B, and the small bound group produces a small to moderate thermal profile 630B. The generation of three thermal profiles is indicative of detectable binding between ligands α and proteins A, and the relative sizes of the thermal profiles (e.g., when compared with previously-established measurement data) can be used to determine the relative strength of the binding event (i.e., relatively small thermal profile 630B indicates weak binding, whereas a relatively strong thermal profile 630B indicates relatively strong binding).

A third example is indicated in FIG. 7(C), where a strong binding event produces an empty unbound ligand group 301C, an empty unbound protein group 303C, and a large bound ligand/protein group 305C. The resulting thermal profiles are indicated in FIG. 8(C), where the empty unbound ligand group generates a flat thermal profile 610C, the empty unbound protein group generates a flat thermal profile 620C, and the large bound group produces a large thermal profile 630C. The generation of only one detectable thermal profile is indicative that substantially all of the ligands and proteins are involved in bound pairs, thereby indicating a strong binding event.

The examples above have all implicitly assumed equal concentrations of ligand and protein. It should be obvious to ones skilled in the arts how to use the same methods when the ratio of concentrations of ligand to protein is small.

Returning to FIG. 2, according to an embodiment of the present invention, after the occurrence of a binding/non-binding event for a first ligand/protein pair, the test channel is “flushed” or otherwise cleansed of residual proteins and ligands (block 250), and then the process is restarted with the injection of a new protein/ligand pair (indicated by arrow between block 250 and block 210). As indicated in FIG. 3(F), the flushing process is performed, for example, using a fluid source 330 coupled to a fluid (e.g., water) source to inject the fluid into receptor well 111, thereby generating a flow of fluid that pushes ligands a and proteins A into sump 112, from which these molecules are removed from channel 110. Flow can be induced using a pressurized input or using EOF generated by the integrated electrodes. Accordingly, by providing a suitable delivery system, test channel 110 can be utilized to test multiple protein/ligand pairs in series. Alternatively, as set forth below, test channel 110 may be used only once (e.g., in conjunction with a massively parallel arrangement), thereby obviating the need for the flushing process. In yet another alternative embodiment, upon completing the binding event detection test, the substrate upon which test channel 110 is formed and/or the injection nozzles of the dose delivery system are repositioned such that the nozzles become aligned with the receptor area of an unused test channel (i.e., either located near the used test channel on the same substrate/unit, or formed on a separate substrate/unit), and then the binding event detection process is restarted.

While the embodiments described above can be used to identify binding events under ideal circumstances, it may not be practical under conditions requiring high throughput and sub-nanoliter sized molecule doses. Also, the arrival time and signal magnitude may not be well known a priori for all ligands. Furthermore, the transport can depend strongly on the absolute values of relatively uncontrolled parameters such as temperature, pH, electric field, etc. Under these circumstances, separation of the molecular groups may be insufficient to identify two or three distinct groups. Further, the amount of material being detected under such circumstances is very small, so even using the absorption enhancing mechanisms (e.g., an etalon) and highly sensitive bolometric detection, as discussed above, it may not be possible to reliably detect the individual groups.

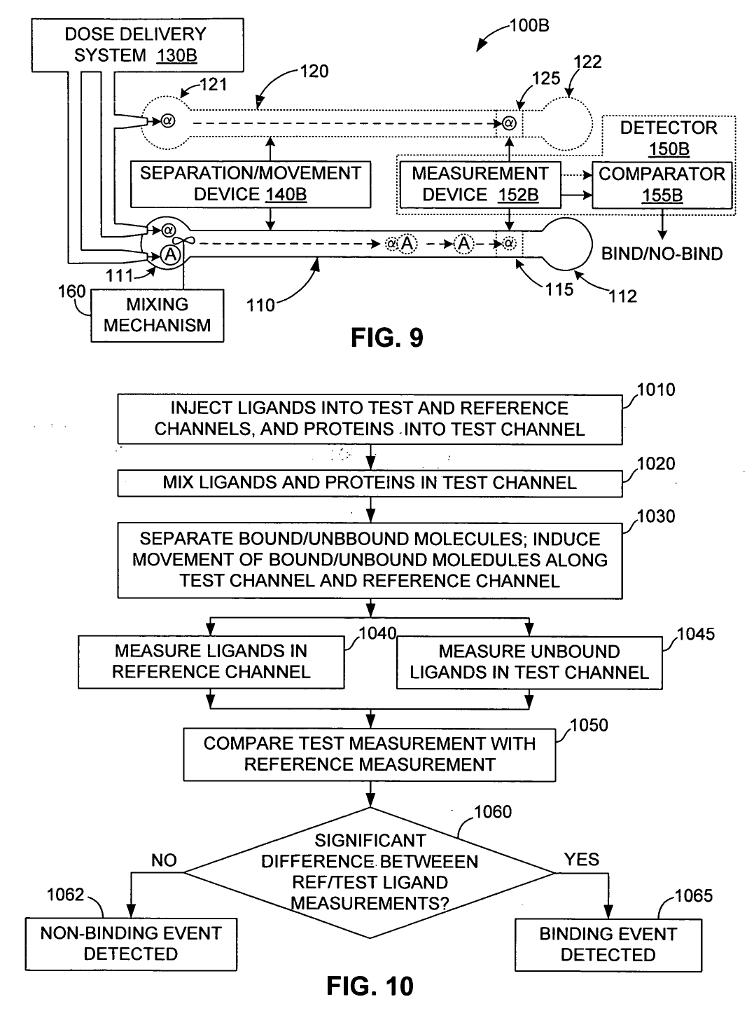

FIG. 9 is a simplified schematic diagram depicting an apparatus 100B for detecting binding events using two channels according to another embodiment of the present invention that facilitates common mode rejection of noise and tolerance to parameter variations as well as the use of smaller doses and higher throughput than the single channel embodiments described above. Apparatus 100B generally includes a test (first) channel 110, a reference (second) channel 120, an optional dose delivery system 130B, a molecular separation movement-inducing device 140B, and a detection device (detector) 150B.

Test channel 110 and reference channel 120 are fabricated in close proximity on a substrate using the fabrication techniques mentioned above, and in one embodiment are substantially the same size, and fabricated in the parallel, side-by-side arrangement depicted in FIG. 9. Test channel 110 includes receptor well 111, sump 112, and intermediate measurement location 115 that function essentially as described above with reference to apparatus 100 and 100A. Reference channel 120 includes a receptor well 121, a sump 122, and a second location 125 that function in the manner described below.

Similar to the previous embodiment, delivery system 130B transports a predetermined dose (first plurality) of ligands a and a predetermined dose of proteins A to receptor well 111 of test channel 110. In addition, delivery system 130B also transports a predetermined dose (second plurality) of ligands a to receptor well 121 of reference channel 120. Mixing mechanism 160, which is operably coupled to receptor well 111, functions as described above to agitate or otherwise intermix ligands a and proteins A in test channel 110 (no mixing is necessary in reference channel 120), but can be optionally included nonetheless to ensure identical timings of the ligands in both channels except for the effects of binding.

Molecular separation/movement device 140B functions to apply a suitable motive force to test channel 110 that induces movement of unbound proteins A and ligands α, or bound protein/ligand pairs, along test channel 110. Separation/movement device 140B also induces movement of ligands α along reference channel 120. In one embodiment, device 140B induces electrophoretic separation/movement. As described above, this motive force causes smaller ligands α to separate from unbound proteins A and bound protein/ligand pairs, and to move along channel 110 from receptor well 111 toward sump 112 at a rate that is similar to the ligands a moving along reference channel 120.

Detection device 150B includes a measurement device 152B and a comparator 155B. Measurement device 152B is arranged to detect ligands a moving past location 115 of test channel 110 and moving past location 125 of reference channel 120 in a manner similar to that described above. Comparator 155B receives measurement data from measurement device 152B, and compares the measured values received from test channel 110 with reference data received from reference channel 120. Based on this comparison, using the methods described below, the present invention facilitates determining the extent to which binding has occurred in a reliable and economical manner.

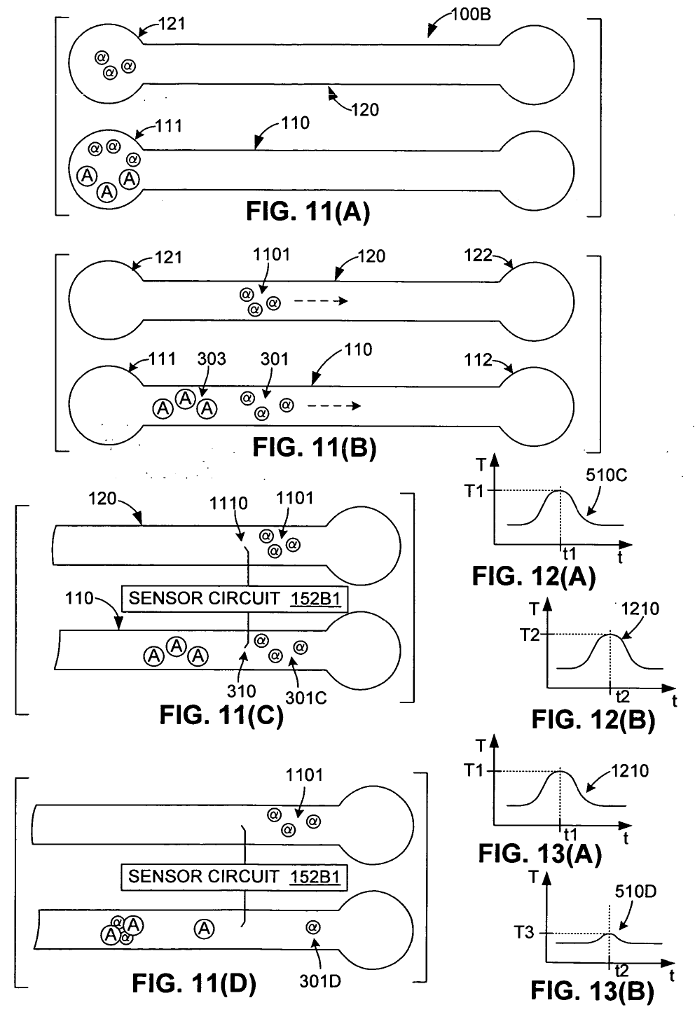

FIG. 10 is a flow diagram showing a generalized method for detecting binding events between two or more molecules according to another embodiment of the present invention. For convenience, the method depicted in the flow diagram of FIG. 10 is described below with reference to FIGS. 11(A) through 11(D), which depict portions of apparatus 100B (described above). Note that the method illustrated by the flow diagram of FIG. 10 is not necessarily restricted to apparatus 100B.

Referring to the upper portion of FIG. 10 and FIG. 11(A), the method begins by injecting and/or mixing first and second molecules (e.g., ligands and proteins) into receptor well 111 of test channel 110, and injecting first molecules (ligands) into receptor well 121 of reference channel 120 (block 1010) using methods similar to those described above. Note that the dose injected into receptor well 121 is substantially the same size (i.e., substantially the same number of ligands α) as that injected into test channel 110. Test channel 110 is then subjected to mixing, for example, by convection and interdiffusion when the second dose is dropped onto the first dose (block 1020). It is also possible to premix ligand and protein in an antechamber before shearing the flow through an orifice during dosing onto the separation array.

As indicated in FIG. 11(B), the mixed molecules in test channel 110 and ligands in reference channel 120 are then induced to move along the respective channels (block 1030; FIG. 10). In one embodiment, electrophoretic movement of ligands a along reference channel 120 at a first rate is initiated by activating a suitable electric field source associated with separation/movement device 140B (FIG. 9). This field source also produces electrophoretic separation of unbound ligands α from unbound proteins A and bound protein/ligand pairs in test channel 110 in the manner described above such that the smaller unbound ligands a move substantially as a group 301 along channel 110 toward sump 112 at a rate that is substantially equal to the movement rate of ligands α along reference channel 120 (i.e., ahead of unbound proteins A and bound protein/ligand pairs). Note that the interaction of ligands a with proteins A and bound pairs may delay the unbound ligand group moving along test channel 110 relative to the ligand group moving along reference channel 120.

As indicated in blocks 1040 and 1045 of FIG. 10, the ligands a moving along channels 110 and 120 are then measured using, for example, stationary probes 310 and 1110 that are respectively located at second locations 115 and 125, and are coupled to a suitable measurement device (e.g., a sensor circuit 150B1, as indicated in FIG. 11(C)). As discussed above, sensor circuit 150B1 takes photothermal measurements that are enhanced by transmitting radiant energy into test channel 110 and reference channel 120, and further enhanced by repeatedly passing the radiant energy back and forth through the channels using an etalon in the manner described above.

The test channel and reference channel measurements are then compared to determine whether a binding event or a non-binding event has occurred between the ligands a and proteins A in test channel 110 (block 1050). In one embodiment, this comparison involves calculating a percentage difference (that is, the difference normalized by the reference signal peak or area) between the test and reference channel measurements, and then determining whether the calculated difference is significant (block 1060). As depicted in FIGS. 11(C), 12(A) and 12(B), when a non-binding event has occurred, substantially equal sized groups of ligands a pass stationary probes 310 and 1110 at approximately the same time, thereby generating similar temperature profiles 510C and 1210 (FIGS. 12(A) and 12(B), respectively). These substantially equal temperature profiles indicate that substantially all of the ligands located in test channel 110 remain unbound, thereby resulting in a non-binding event determination (block 1062). Conversely, as depicted in FIGS. 11(D), 13(A) and 13(B), when a binding event has occurred, the resulting temperature profile 1110 (FIG. 13(A)) is substantially more pronounced than a temperature profile 510D (FIG. 13(B)). These substantially different temperature profiles indicate that substantially all of the ligands located in test channel 110 are bound to corresponding proteins A, thereby resulting in a binding event determination (block 1065). Intermediate levels indicate quantitatively different levels of binding for the given mixing concentrations, mixing times, temperature, etc.

As in the previous embodiments, after determining the occurrence of a binding/non-binding event, test channel 110 and reference channel 120 may be “flushed” or otherwise cleansed of residual proteins and/or ligands, and then the process is restarted with the injection of a new protein/ligand pair.

To this point the present invention has been described with reference to simplified embodiments including one or two channels and associated dose delivery systems that involve the testing of a single protein/ligand pair. The following embodiments illustrate how the present invention can be modified to perform binding event detection on a large scale.

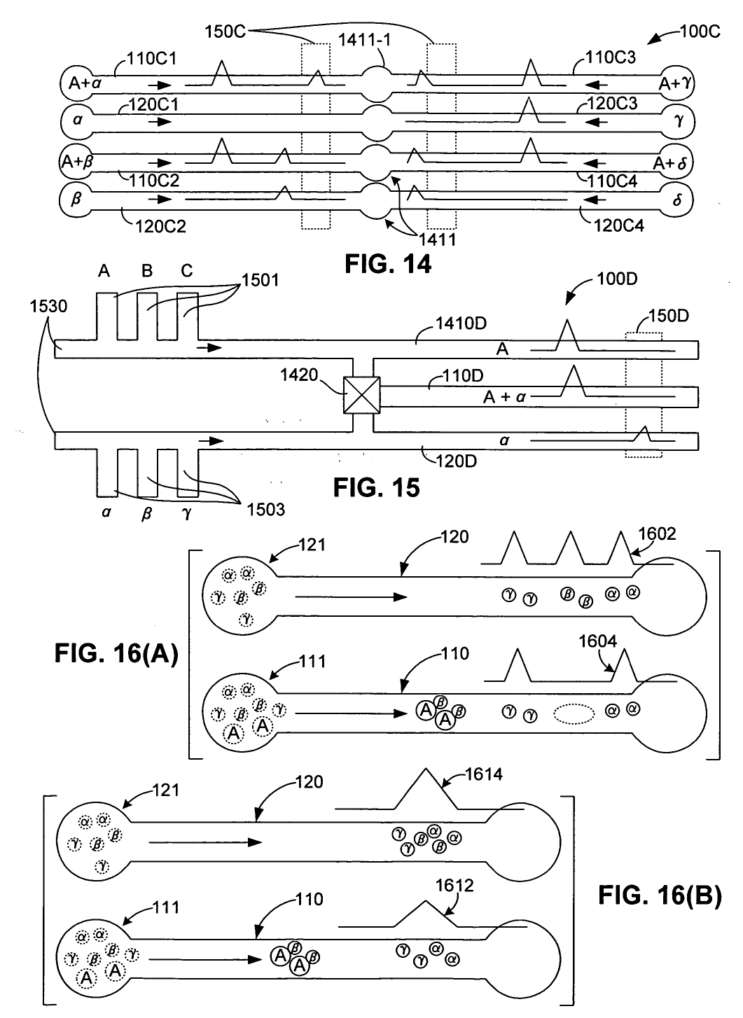

FIG. 14 is a simplified diagram showing an apparatus 100C according to another embodiment of the present invention. Apparatus 100C includes multiple test channels 110C1 through 110C4 and multiple reference 120C1 through 120C4, wherein each pair of test and reference channel receives a corresponding ligand α, β, γ, and δ, and tests the corresponding ligand against protein A using a measurement device 150C according to the methods set forth above. For example, test channel 110C1 receives doses of ligand α and protein A, and reference channel 120C1 receives a dose of ligand α. In contrast, test channel 110C2 receives doses of ligand β and protein A, and reference channel 120C2 receives a dose of ligand β. In addition, centrally located sump regions 1411 are shared by corresponding pairs of channels. For example, test channels 10C1 and 110C3 share sump region 1411-1. By distributing protein A to all of the channels in this manner, it is understood that binding event detection can be performed on a massively parallel scale.

FIG. 15 is a simplified diagram showing an apparatus 100D according to another specific embodiment. Apparatus 100D a first set of inlet ports 1501 selectively inject proteins into a first channel 1510D, and a second set of inlet ports 1503 selectively inject ligands into a reference channel 120D. Both channels 1510D and 120D communicate with a mixing (valve) mechanism 1520 that passes predetermined portions of the injected proteins and ligands into test channel 110D. Subsequent binding event detection is then performed using the methods described above. After a particular test, the channels are flushed, for example, by injecting water through end ports 1530 of channels 1510D and 120D, respectively. Accordingly, apparatus 100D facilitates sequential testing of multiple proteins and ligands using a single set of channels.

According to another aspect of the present invention, the apparatus and methods described may be used to detect binding events in a highly efficient manner by mixing multiple ligands with a subject protein in a single channel, detecting binding of at least one of the ligands with the protein, and then performing separate tests to identify the binding ligand(s). For example, as indicated in FIGS. 16(A) and 16(B), multiple non-interacting ligands (e.g., α, β, γ) are mixed with a subject protein A. If one of these ligands binds with protein A (e.g., ligand β, as indicated in the figures), then the resulting absence of the binding ligand can be detected using the methods described above, and one or more additional separation processes can be used to identify the specific binding ligand (if necessary). For example, as shown in FIG. 16(A), if ligands α, β, γ have different sizes and separate as they move down channels 110 and 120, then the absence of ligand β is detectable by comparing the associated thermal profiles 1602 and 1604, which are superimposed over each channel. Alternatively, as indicated in FIG. 16(B), if ligands α, β, γ do not separate significantly as they move down channels 110 and 120, then the absence of ligand β is detectable by comparing the reference channel thermal profile 1612 with the smaller test channel thermal profile 1614 (here, a follow-up procedure, e.g., repeating the test with one or more of ligands α, β, γ separately tested in associated channel pairs may be necessary to identify β as the binding ligand. Of course, in addition to the two-channel testing method indicated in FIGS. 16(A) and 16(B), the single channel testing methods described above may also be used. The benefit of testing multiple ligands is to reduce the number of test iterations required to identify a relatively small number of binding ligands from a relatively large library of ligands. For example, in the case of three ligands per channel, instead of executing up to thirty single-ligand iterations to identify one binding ligand from a library of thirty ligands, the present method requires, perhaps twelve iterations (i.e., up to ten three-ligand iterations to identify the group of three ligands including the binding ligand, then two extra single-ligand iterations to disambiguate in the rare event that dispersed ligands spatially overlap in the detection zone of one of the channels). Of course, further efficiencies may be achieved by combining a larger number of ligands per iteration, and/or performing the multi-ligand iterations in a massively parallel arrangement.

Although the present invention has been described with respect to certain specific embodiments, it will be clear to those skilled in the art that the inventive features of the present invention are applicable to other embodiments as well, all of which are intended to fall within the scope of the present invention. For example, absorbing molecules that are related to the dosed samples (analytes) may have to be taken into account in the detection determination, but the inventors believe it is safe to restrict those measurement components to a constant set that is compatible with the subject (e.g., protein A) molecules (i.e., because the library of ligands also has to be compatible with the chemistry of these subject molecules). Therefore, all channels would have a measurement peak or peaks that would arise from the analytes, but these peaks could be known to the detection system and eliminated from the measurement data output to the user. Alternatively, a third channel just including the “protein A” molecules (and associated analytes) may also be included in the test arrangement. The detected signal from this third channel could be used as a mask to block uninteresting signals from the test channel.

1. A method for detecting binding events between first molecules and second molecules, the method comprising:
inducing movement of a mixture containing both a first plurality of the first molecules and a plurality of the second molecules along a first channel from a first location toward a second location, and for inducing movement of a second plurality of the first molecules along a second channel from a third location toward a fourth location;
measuring a first amount of said first molecules passing the second location during a first time period, measuring a second amount of said first molecules passing the fourth location during a second time period; and
determining an occurrence of said binding event between the first and second molecules by comparing the first and second measured amounts.

2. The method according to claim 1, wherein inducing movement further comprises separating respectively unbound ones of the first molecules from unbound ones of the second molecules and bound pairs of first and second molecules in the first channel.

3. The method according to claim 1, wherein inducing movement comprises applying a moving force such that the unbound ones of the first molecules move in the first and second channels at a first rate, and bound pairs of first and second molecules move in the first channel at a second rate different from the first rate.

4. The method according to claim 3, wherein applying the moving force comprises applying an electric field to the first and second channels.

5. The method according to claim 1, wherein measuring comprises photothermally detecting first molecules passing the second location.

6. The method according to claim 5, wherein the first and second channels contain a fluid, and wherein the method further comprises transmitting radiant energy beams into the first and second channels, and measuring temperature changes in the fluid that are generated by heat absorbed from the radiant energy by the first and second molecules.

7. The method according to claim 6, wherein measuring temperature changes comprises placing a bolometer in contact with the fluid contained in the first channel such that the bolometer is positioned outside of the radiant energy beams transmitted into the first channel.

8. The method according to claim 5, wherein measuring the first amount comprises capturing a first temperature profile generated by said first molecules passing the second location,
wherein measuring the second amount comprises capturing a second temperature profile generated by said first molecules passing the fourth location, and
wherein comparing comprises calculating a percentage difference between the first and second temperature profiles.
